(19) United States
(12) Patent Application Publication
(10) Pub. No.: US 2002/0023847 A1
(43) Pub. Date: Feb. 28, 2002

(54) Cleansing system and method using water electrolysis

(76) Inventor: Shinichi Natsume, Shinshiro-shi (JP)

Correspondence Address: Squire, Sanders & Dempsey L.L.P., 600 Hansen Way, Palo Alto, CA, 94304, US

(21) Appl. No.: 09/875,539

(22) Filed: Jun. 6, 2001

Related U.S. Application Data
(60) Provisional application No. 60/213,460, filed on Jun. 23, 2000.

(51) Int. Cl. C02F 1/461

(52) U.S. Cl. 205/687; 205/742; 205/746; 204/252; 204/263; 204/275.1

(57) ABSTRACT

A system for cleansing using an alkaline solution formed in an electrolysis chamber. The system comprises an electrolysis chamber wherein the alkaline solution is formed from an electrolyte, such as salt, and water. At least one pump outputs the alkaline solution onto a object to be cleansed. Further, a second pump may output an acidic solution formed in the electrolysis onto the object to disinfect or sterilize the object.

### PRIORITY REFERENCE TO PRIOR APPLICATION

This application claims the benefit of and incorporates by reference provisional patent application Ser. No. 60/213,460, entitled “Cleansing Equipment Using Water Electrolysis,” filed on Jun. 23, 2000, by inventor Shinichi Natsume.

### TECHNICAL FIELD

This invention relates generally to cleansing systems, and more particularly, but not exclusively, provides a system and method for generating a cleansing solution via water electrolysis.

### BACKGROUND

Conventional detergents may contain several potential pollutants including phosphates, enzymes, flurescers, silicates and sulphates. Phosphates are particularly polluting of the environment because phosphates are a rich source of nutrients for algae. When waste water containing phosphates is deposited into bodies of water, the algae consumes the phosphates and then bloom. When the algae later dies, decomposition of the algae may use up most of the dissolved oxygen in the bodies of water contaminated by the phosphates. The bodies of water may then become uninhabitable to oxygen-dependent life in the water.

Therefore, a new system and method for cleansing without pollutants may be desirable.

### SUMMARY

The present invention provides a cleansing and sterilizing apparatus for cleaning objects such as dishes, medical devices, clothing, etc. The apparatus comprises an electrolysis chamber that uses an ion exchange membrane to separate a cathode section from an anode section of the chamber. Tap water is injected into cathode section and saltwater is injected into the anode section. Electrolysis in the anode section of the electrolysis chamber forms acidic water and HOCl, which has antibacterial and disinfectant properties. In addition, electrolysis in the cathode section of the electrolysis chamber forms alkaline water and sodium hydroxide (NaOH), which has cleansing and reducing properties. The alkaline water and sodium hydroxide is then pumped out of the electrolysis chamber sprayed on objects to be cleansed, such as medical devices (endoscopes, dialysis equipment), dishes, etc. After cleansing, the objects may then be optionally sprayed with the acidic water and HOCl to sterilize or disinfect the objects.

In alternative embodiments of the apparatus, the ion exchange membrane includes an anion exchange membrane, a cation exchange membrane, and a neutral membrane. In another embodiment of the invention, the electrolysis chamber does not include an ion exchange membrane, thereby reducing costs of manufacturing and using the invention.

The present invention further provides a cleansing and sterilizing method. The method comprises injecting tap water into a cathode section of the electrolysis chamber and saltwater into the anode section of the chamber. The electrolysis chamber then applies a negative voltage to the cathode and a positive voltage to the anode. HOCl and highly acidic water then forms in the anode section of the chamber and NaOH and alkaline water forms in the cathode section of the chamber. Pumps then pump the NaOH and alkaline water out of the chamber and spray it on the objects to be cleansed. Afterwards, pumps pump the HOCl and the highly acidic water out of the anode section of the chamber and spray the acidic water and HOCl onto the cleansed objects in order to disinfect the objects.

The system and method may advantageously cleanse and sterilize objects using only saltwater and tap water, thereby preventing water pollution though the use of phosphates and other chemicals.

### BRIEF DESCRIPTION OF THE DRAWINGS

FIG. 1 is a diagram illustrating a cleansing system in accordance with an embodiment of the present invention;

FIG. 2 is a diagram illustrating an alternative embodiment of the cleansing system of FIG. 1;

FIG. 3 is a diagram illustrating an electrolysis chamber for use in the cleansing system of FIG. 1 or FIG. 2;

FIG. 4 is a diagram illustrating an alternative embodiment of an electrolysis chamber for use in the cleansing system of FIG. 1 or FIG. 2; and

FIG. 5 is a diagram illustrating a dishwasher system according to an embodiment of the invention.

### DETAILED DESCRIPTION OF THE ILLUSTRATED EMBODIMENTS

The following description is provided to enable any person skilled in the art to make and use the invention, and is provided in the context of a particular application and its requirements. Various modifications to the embodiments will be readily apparent to those skilled in the art, and the generic principles defined herein may be applied to other embodiments and applications without departing from the spirit and scope of the invention. Thus, the present invention is not intended to be limited to the embodiments shown, but is to be accorded the widest scope consistent with the principles, features and teachings disclosed herein.

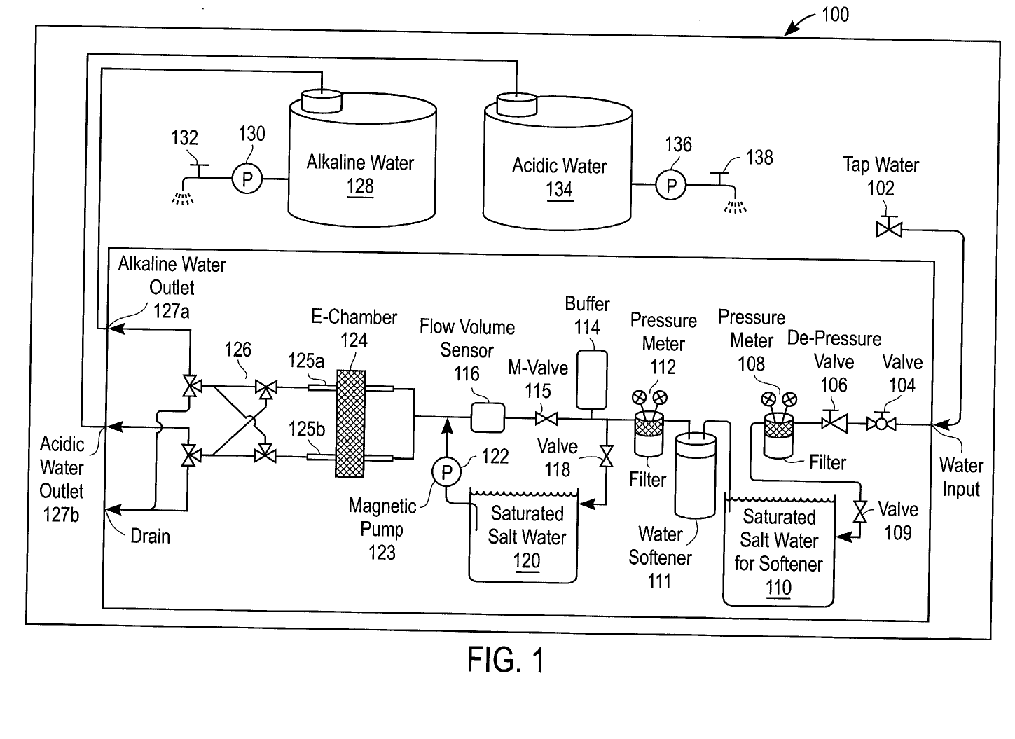

FIG. 1 is a diagram illustrating a cleansing system 100 in accordance with an embodiment of the present invention. System 100 may be used to cleanse and/or sterilize a variety of objects including medical equipment (dialysis machines, endoscopes, etc.) and dishes, etc. Valve 102 is coupled to a water source for inputting water into system 100. Like many other optional items in cleansing system 100, valves 104, 106, 109, 115, and 118 are optional and regulate the flow of liquid throughout system 100. In addition, optional pressure meters 108 and 112, and flow volume sensor 116 measure the pressure and flow of liquid throughout system 100. Before tap water enters electrolysis chamber 124, an optional water softener 111 uses saltwater in saltwater container 110 to soften the tap water so as to remove any calcium or magnesium that might be in the tap water. If calcium or magnesium is present in system 100, the calcium or magnesium may cause clogging of piping within system 100. Further, calcium or magnesium might effect production of cleansing and antibacterial solutions in electrolysis chamber 124. Accordingly, removal of calcium and magnesium from the input tap water also ensures invariable production of cleansing and antibacterial solutions.

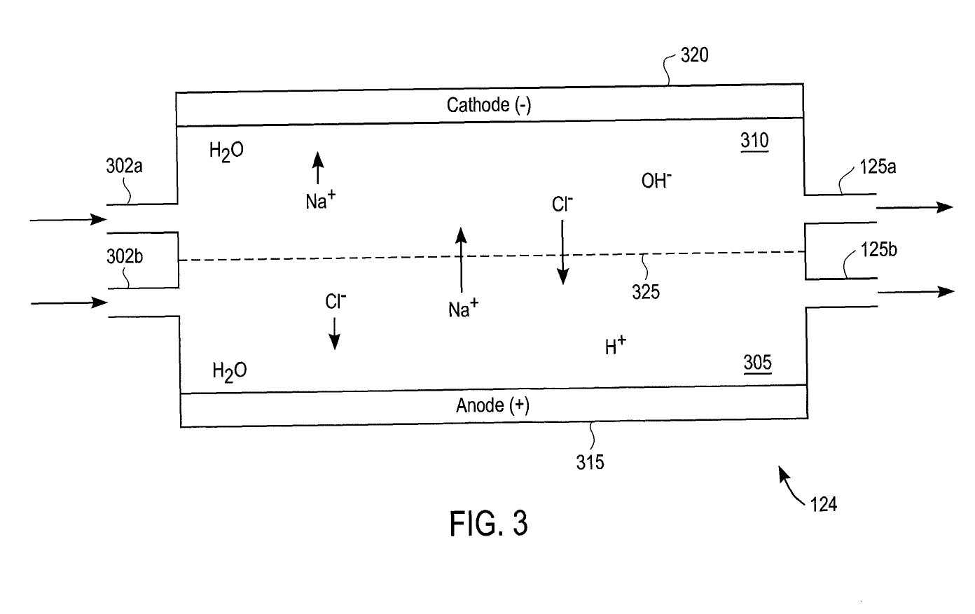

After softening, the softened tap water enters electrolysis chamber 124. In addition, magnetic pump 123 pumps saturated salt water into electrolysis chamber 124. The electrolysis chamber 124 produces an acidic solution containing acidic water with HOCl, and/or an alkaline solution containing alkaline water with NaOH. The acidic water and HOCl has antibacterial or antiseptic properties while the alkaline water with NaOH has cleansing or reducing properties. Production of these solutions within electrolysis chamber 124 will be discussed in further detail in conjunction with FIG. 3. In an alternative embodiment, magnetic pump 123 may pump other compounds, such as potassium chloride or calcium chloride, into electrolysis chamber 124.

After production of alkaline and/or acidic solutions in the electrolysis chamber 124, the alkaline and acidic solutions are separately pumped out of the chamber 124 via piping 125a and 125b respectively. Piping 125a and piping 125b are coupled to alkaline water outlet 127a and acidic water outlet 127b respectively, via optional assembly 126. Under default conditions, the alkaline solution flows out of piping 125a, across assembly 126 and into alkaline solution outlet 127a. In addition, the acidic solution flows out of piping 125b through assembly 126 and into acidic solution outlet 127b. However, if the polarity in the electrolysis chamber 124 is reversed in order to reverse an accumulation of minerals within electrolysis chamber 124, then the alkaline solution will flow out of piping 125b instead of 125a and acidic solution will flow out of piping 125a instead of piping 125b. Assembly 126 then reroutes the alkaline solution so that the alkaline solution enters alkaline solution outlet 127a and reroutes the acidic solution so that the acidic solution enters acidic solution outlet 127b.

Alkaline solution (containing alkaline water and NaOH) flows from alkaline solution outlet 127a to alkaline solution storage tank 128. Acidic solution (containing acidic water and HOCl) flows from acidic solution outlet 127b to acidic solution storage tank 134. During a cleansing process, pump 130 pumps alkaline water and HOCl out of alkaline solution storage tank 128 and through spigot 132 onto objects to be cleansed. The alkaline water and NaOH cleanse the objects. Pump 136 then pumps acidic water and HOCl out of acidic solution storage tank 134 and through spigot 138 onto objects to be sterilized/disinfected.

In an alternative embodiment of system 100, system 100 does not include an alkaline solution storage tank 128 and an acidic solution storage tank 134. Accordingly, during a cleansing process, alkaline solution flows directly from alkaline solution outlet 127a onto the object and acidic solution flows directly from acidic solution outlet 127b onto the object.

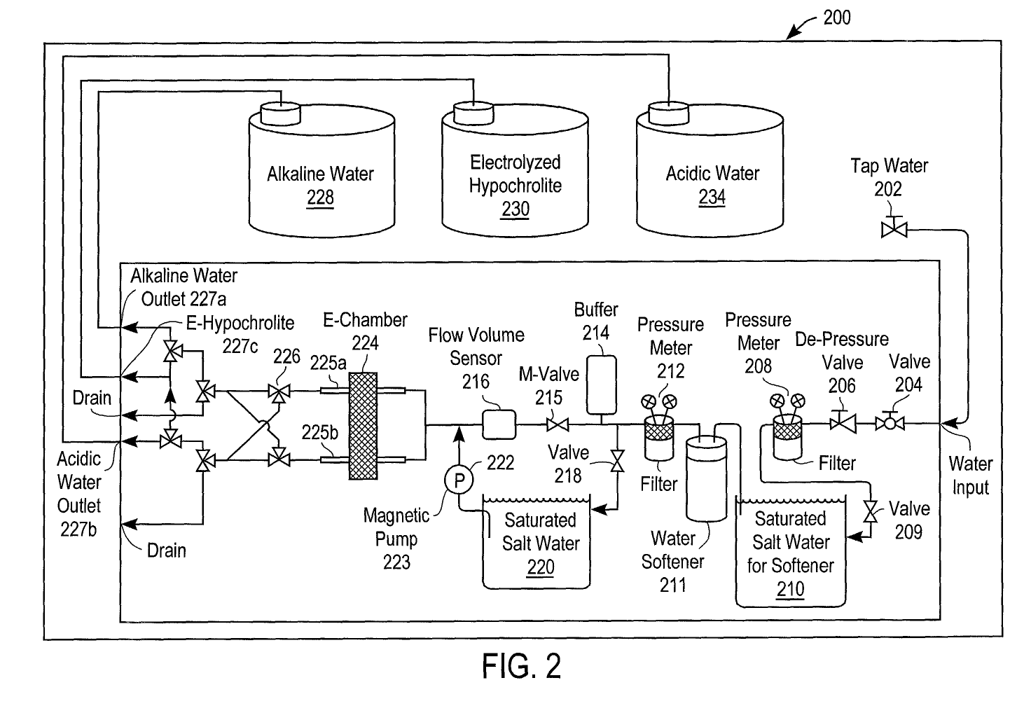

FIG. 2 is a diagram illustrating a cleansing system 200 according to an alternative embodiment of the invention. System 200 may be used to cleanse and/or sterilize a variety of objects including medical equipment (dialysis machines, endoscopes, etc.) and dishes, etc. As in system 100 (FIG. 1), system 200 comprises multiple valves to regulate the flow of liquids within system 100 and multiple pressure meters to measure pressure within system 200. System 200 further comprises an optional water softener 211 that uses saturated saltwater 210 for softening input tap water.

Electrolysis chamber 224 received saturated saltwater from saltwater container 220 and softened tap water from softener 211. An anode section of the electrolysis chamber 224 produces an acidic solution containing acidic water and HOCl, which as antibacterial properties. A cathode section of the electrolysis chamber 224 produces an alkaline solution containing alkaline water and NaOH, which has reducing or cleansing properties. The alkaline water and NaOH is then collected in alkaline solution tank 228. Acidic water and HOCl is collected in acidic solution tank 234. In addition, a mixture of acidic solution and alkaline solution is collected in electrolyzed hypochrolite tank 230. The mixture of acidic solution and alkaline solution in hypochrolite tank 230 has a combination of reducing and antibacterial properties and is commonly used for cleansing vegetables. Pumps and spigots (not shown) can then spray the liquids from alkaline solution tank 228, hypochrolite solution tank 230, and acidic solution tank 234 onto objects to cleanse and disinfect the objects.

FIG. 3 is a diagram illustrating the electrolysis chamber 124 of system 100 (FIG. 1). Electrolysis chamber 124 is identical to electrolysis chamber 224 of system 200 (FIG. 200). Electrolysis chamber 124 comprises an anode 315 and a cathode 320. Electrolysis chamber 124 further comprises a neutral ion exchange membrane 325, which allows both negative and position ions to flow between cathode section 310 and anode section 305.

Saltwater and tap water enter electrolysis chamber 124 via piping 302a and 302b. The salt (NaCl) dissolves into Na⁺ and Cl⁻. During electrolysis, the Na⁺ moves towards the cathode 320 and Cl⁻ moves towards the anode 315. As ion membrane 325 is neutral, both Na⁺ and Cl⁻ can cross the membrane 325. In anode section 305, chlorine and water react to form HOCl, which has antibacterial properties. Specifically, Cl₂+H₂O→HOCL+H⁺+Cl⁻ to form an acidic solution having a pH in the range of 2-4, with 2.3 being typical. The acidic solution then exits the electrolysis chamber 124 via piping 125a.

In the cathode section 310, sodium bonds with hydroxyl groups to form sodium hydroxide. Specifically, Na⁺+OH⁻→NaOH to form an alkaline solution in cathode section 310 having a pH in the range of 10-12, with 12 being typical. The alkaline solution then exits cathode section 310 via piping 125b. Chlorinated substances, such as, potassium chloride, calcium chloride, etc., may also be introduced into the cathode section, in place of NaCl to produce reduced cleansing water. Alternatively, chlorinated substances may be introduced into the cathode section in addition to the NaCl introduced into the chamber 124 so that the chlorinated substance(s) and NaOH are present to further increase cleansing efficacy.

Further, a cleansing agent injected into the cathode and/or anode section of the chamber 124 will enhance activity and cleansing power without changing components of the cleansing agent. In addition, adding agents may be added to the cathode or anode sections to enhance the cleansing efficacy of electrolyzed water.

In an alternative embodiment of electrolysis chamber 124, membrane 325 is a cation membrane, which only allows cations (i.e. Na⁺) to pass through. Accordingly, saltwater is only introduced into anode section 305. Na⁺ crosses membrane 325 into cathode section 310, but Cl⁻ is unable to cross the membrane 325 into cathode section 310. Using the cation membrane in electrolysis chamber 124 leads to a higher concentration of sodium ions in the cathode section 310 and chlorine ions in the anode section 305 as compared to using a neutral membrane. This in turn leads to a higher alkaline solution in the cathode section and a higher acidic solution in the anode section, causing improved cleansing and sterilizing in system 100.

In addition, a cleansing agent may be introduced into the cathode section 310 of the alternative embodiment of electrolysis chamber 124. Negative ions of the cleansing agent cannot pass through the cation membrane 325 and therefore, the cleansing agent ions remain in the cathode section 310 of electrolysis chamber 124, leading to increased cleansing power of the alkaline solution in the cathode section 310.

In a second alternative embodiment of electrolysis chamber 124, membrane 325 is an anion membrane, which only allows anions to pass through membrane 325. Cleansing liquid is injected in anode section 305 and saltwater is injected into cathode section 310. Cl⁻, attracted to the anode 315, moves across the anion membrane 325 into anode section 305. Further, cations from the cleansing liquid cannot pass through the anion membrane and therefore remain in the anode section 305. Accordingly, HOCl is formed in anode section 305 and NaOH is formed in cathode section 310. As a result, the cleansing liquid is highly acidic since the cleansing liquid is introduced to the anode section 305.

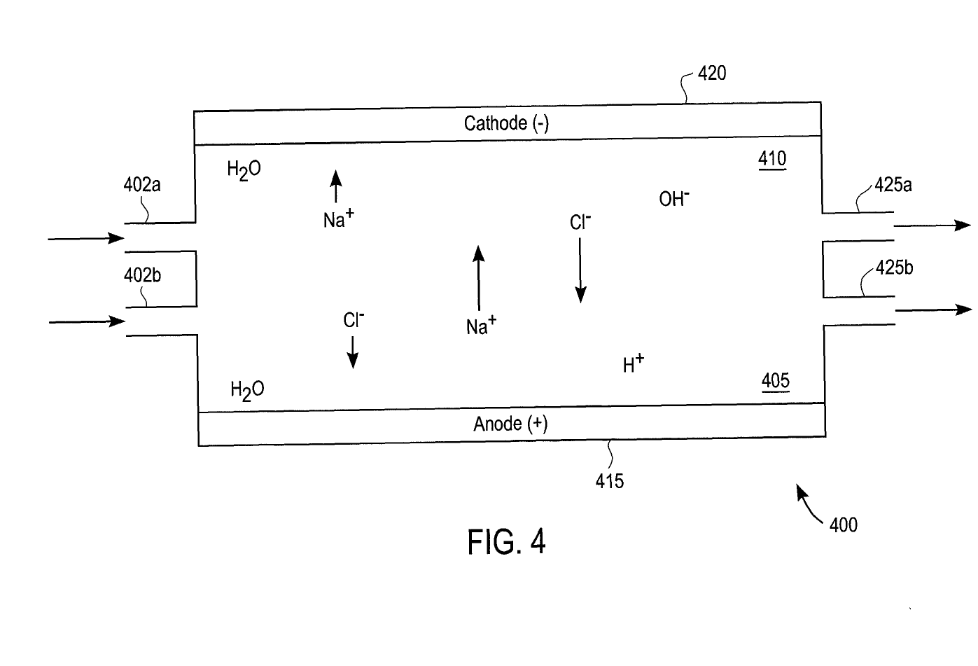

FIG. 4 is a diagram illustrating an alternative embodiment of an electrolysis chamber 400 for use in cleansing system 100 (FIG. 1) or cleansing system 200 (FIG. 2). Electrolysis chamber 400 comprises cathode 420 and anode 415. Unlike electrolysis chamber 124, electrolysis chamber 400 does not have an ion exchange membrane.

Saltwater and tap water enter electrolysis chamber 400 via piping 402a and 402b. As there is no ion membrane, the saltwater and tap water may be mixed together and enter through either piping 402a or 402b or both piping 402a and 402b. Alternatively, electrolysis chamber 400 may have only a single input pipe for inputting a mixture of saltwater and tap water.

The salt (NaCl) dissolves in Na⁺ and Cl⁻. During electrolysis, the Na⁺ moves towards the cathode 320 and Cl⁻ moves towards the anode 315. In anode section 305, chlorine and water react to form HOCl, which has antibacterial properties. Specifically, C₂+H₂O→HOCL+H⁺+Cl⁻ to form an acidic solution having a pH in the range of 2-4. The acidic solution then exits the electrolysis chamber 400 via piping 425a.

In the cathode section 410, sodium bonds with hydroxyl groups to form sodium hydroxide. Specifically, Na⁺+OH⁻→NaOH to form an alkaline solution in cathode section 410 having a pH in the range of 10-12. The alkaline solution then exits cathode section 410 via piping 425b. The alkaline solution from electrolysis chamber 400 is generally slightly less alkaline than the alkaline solution from electrolysis chamber 124. Similarly, the acidic solution from electrolysis chamber 400 is generally only slightly less acidic than acidic solution from electrolysis chamber 124. Accordingly, it may be preferable to use a non-membrane electrolysis chamber 400 in system 100 or system 200 since there is a reduction in cost of manufacturing and using system 100 or system 200 due to the lack of a membrane with only a slight decrease in acidity of the acidic antibacterial solution and a slight decrease in alkalinity of the alkaline cleansing solution. Further, a cleansing agent may be introduced into chamber 400, thereby enhancing activity and efficacy of the electrolyzed water.

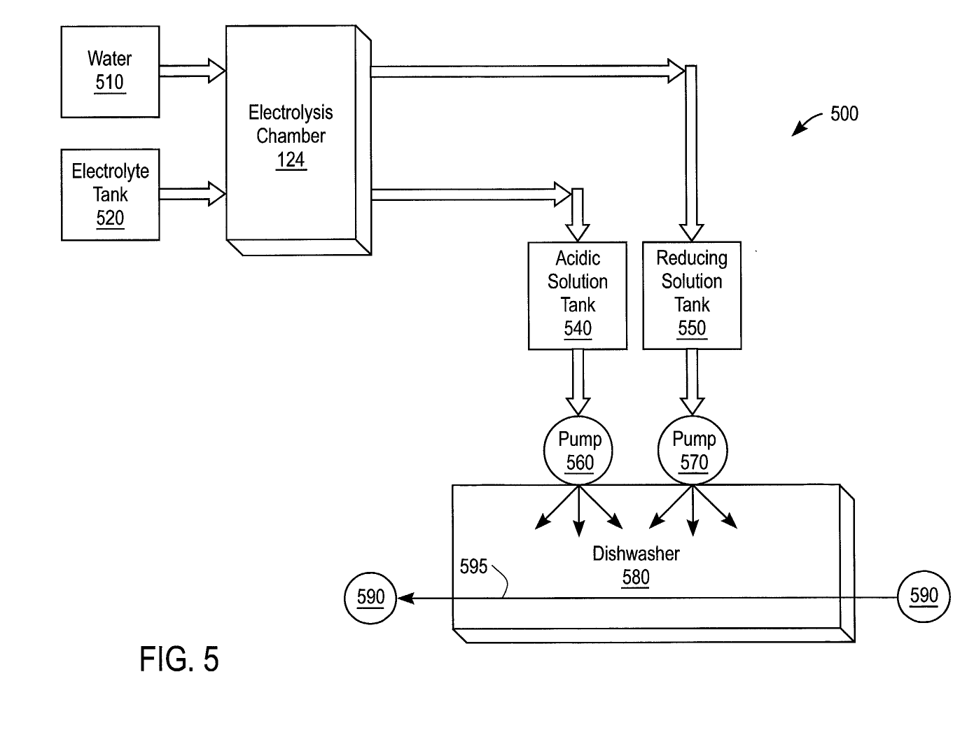

FIG. 5 is a diagram illustrating a dishwasher system 500 according to an embodiment of the invention. A pump (not shown) injects tap water from tap water tank 510 into a cathode section of electrolysis chamber 124. A second pump (not shown) injects electrolytes, such as salt, potassium chloride, or calcium chloride, from electrolyte tank 520 into an anode section of electrolysis chamber 124. Within the electrolysis chamber 124, as discussed in conjunction with FIG. 3, salt dissolves into Na⁺ and Cl⁻ and forms NaOH in an alkaline solution in the cathode section and HOCl in an acidic solution in the anode section. Pumps (not shown) then pump the acidic solution from the electrolysis chamber 124 into acidic solution tank 540 and the alkaline solution from electrolysis chamber 124 into reducing solution tank 550.

During a cleansing cycle, a dish 590 passes through dishwasher 580 on a conveyor belt 595. As the dish 590 passes through the dishwasher 580, pump 570 sprays the dish 590 with the alkaline solution from reducing solution tank 560. The alkaline solution cleanses the dish 590. As the dish 590 travels further in the dishwasher 580, pump 560 sprays the dish 590 with acidic solution from acidic solution tank 540, thereby disinfecting the dish 590. The dish 590 then exits the dishwasher 580 cleansed and disinfected. Alternatively, the dish 590 may be moved manually through dishwasher 580 or the dish 590 may be stationary within dishwasher 580. Further, any object may be cleansed in system 500 as long as the system 500 is scaled appropriately to the size of the object. Examples of possible objects for cleansing in system 500 include silverware, medical devices, clothing, etc.

The foregoing description of the preferred embodiments of the present invention is by way of example only, and other variations and modifications of the above-described embodiments and methods are possible in light of the foregoing teaching. For example, potassium chloride may be used as an electrolyte in place of salt. The embodiments described herein are not intended to be exhaustive or limiting. The present invention is limited only by the following claims.

What is claimed is:

1. A method, comprising:
(a) inputting an electrolyte and water into an electrolysis unit;
(b) generating, in the electrolysis unit, an alkaline solution for cleansing and an acidic solution for disinfecting;
(c) outputting at least one of alkaline solution or acidic solution from the electrolysis unit onto an object.

2. The method of claim 1, further comprising outputting either a solution not output in (c).

3. The method of claim 1, wherein the electrolyte is NaCl.

4. The method of claim 1, further comprising softening the water before inputting the water into the electrolysis unit.

5. The method of claim 1, wherein the electrolysis unit comprises a cation membrane located within the electrolysis unit so as to divide the unit into an anode section and a cathode section.

6. The method of claim 5, wherein the electrolyte is inputted into the anode section and water is inputted in the cathode section.

7. The method of claim 6, further comprising inputting a cleansing agent into the cathode section.

8. A system, comprising:
means for inputting an electrolyte and water into an electrolysis unit;
means for generating, in the electrolysis unit, an alkaline solution for cleansing and an acidic solution for disinfecting;
means for outputting at least one of the alkaline solution or the acidic solution from the electrolysis unit onto an object.

9. A system, comprising:
an electrolysis unit, the electrolysis unit using water and an electrolyte to generate an alkaline solution for cleansing and an acidic solution for disinfecting;
a first pump unit coupled to the electrolysis unit to pump out at least one of the alkaline solution or the acidic solution onto an object.

10. The system of claim 9, further comprising a second pump unit coupled to the electrolysis unit to pump out either the alkaline solution or the acidic solution that is not pumped out by the first pump unit.

11. The system of claim 10, further comprising an alkaline solution tank to store the alkaline solution and an acidic solution tank for storing the acidic solution.

12. The system of claim 9, wherein the electrolyte is NaCl.

13. The system of claim 9, further comprising a water softener for softening the water before input into the electrolysis unit.

14. The system of claim 9, wherein the electrolysis unit further comprises an ion membrane located within the electrolysis unit so as to divide the unit into an anode section and a cathode section.

15. The system of claim 14, wherein the ion membrane includes an anion membrane.

16. The system of claim 14, wherein the ion membrane includes a cation membrane.

17. The system of claim 16, wherein the electrolyte is inputted into the anode section and water is inputted into the cathode section.

18. The system of claim 17, wherein a cleansing agent is inputted into the cathode section.
